(19) United States
(12) Patent Application Publication
(10) Pub. No.: US 2020/0306126 A1
(43) Pub. Date: Oct. 1, 2020

(54) PALM WRAP DEVICE

(71) Applicant: CATHERINE CARVALHO, CHARLOTTE, NC (US)

(72) Inventor: CATHERINE CARVALHO, CHARLOTTE, NC (US)

(21) Appl. No.: 16/827,223

(22) Filed: Mar. 23, 2020

Related U.S. Application Data
(60) Provisional application No. 62/825,893, filed on Mar. 29, 2019.

(51) Int. Cl. A61H 7/00; A41D 13/08; A61F 13/10

(52) U.S. Cl. CPC A61H 7/003; A41D 13/088; A61H 2201/1695; A61H 2201/1692; A61F 13/108

(57) ABSTRACT

A palm wrap device to assist with deep tissue massages provides tension relief by serving as an alternative to using the thumb and is easy to use and remove. The device contains a top surface; a bottom surface; a broad end; a first protruding end; a second protruding end; a first fastener, second fastener, third fastener, and fourth fastener; a palm pocket; one or more palm wedges; and an optional wrist support band.

### REFERENCE TO RELATED APPLICATIONS

This application claims the benefit of U.S. Provisional Application No. 62/825,893, filed on Mar. 29, 2019, which is hereby incorporated in its entirety.

### FIELD OF THE INVENTION

The present invention relates to deep tissue massaging devices. More specifically, the invention pertains to a palm wrap device configured to provide deep tissue massage. Additionally, the present invention relates to a method for preventing wrist sprains and arthritis caused from overuse of the thumb during a deep tissue massage.

### BACKGROUND OF THE INVENTION

The following description is not an admission that any of the information provided herein is prior art or relevant to the present invention, or that any publication specifically or implicitly referenced is prior art. Any publications cited in this description are incorporated by reference herein. Where a definition or use of a term in an incorporated reference is inconsistent or contrary to the definition of that term provided herein, the definition of that term provided herein applies and the definition of that term in the reference does not apply.

Often, a masseuse does not offer deep tissue massages as a service because of the long-term effects and impact on the hands. Specifically, the thumb itself and joints supporting the thumb cannot endure providing the needed pressure associated with deep tissue massages on a daily basis. Such anticipated effects include joint arthritis from overuse of the thumbs, chronic inflammation, and the potential development of debilitating conditions such as carpal tunnel syndrome or other contact repetitive stress injuries. As a result, a masseuse may only be able to engage in deep tissue massages for a limited time due to the potential for such repetitive stress injuries.

Yet many people suffer from chronic pain due to various types of disease. This will lead to stiffness that becomes unbearable and difficult to resolve without the assistance of routine deep tissue massages. If no action is taken, the pain cycle worsens, the muscles become shorter, and movement becomes more difficult and more restricted.

Currently, most hand wraps only function to protect a human user's hand during physical activities and to support the wrist when worn. These hand wraps also vary as to the design related to how much of the hand and fingers are exposed. However, the present hand wraps cannot be worn during the massaging process allowing the masseuse to give a deep tissue massage while preventing pain and arthritis in the masseuse's hands. Further, hand wraps currently on the market do not have the capability for a user to insert additional objects that would enable the user to expand the use of the hand wrap.

For the foregoing reasons, a need exists for a device that provides the support and protection of a hand wrap while offering the added benefit of assisting a masseuse with preventing the negative physical effects associated with giving a deep tissue massage.

### SUMMARY OF THE INVENTION

The present invention relates to a palm wrap device that is configured to assist a masseuse or other user in the physical act of deep tissue massaging. The device comprises a top surface and a bottom surface with a palm pocket for removably inserting one or more palm wedges; a first protruding tab and a second protruding tab positioned around the thumb and attached to a broad end by a first, second, third, and fourth fastener; or any combination thereof; and an optional wrist support band for additional wrist support. In one embodiment, the material of construction for the device comprises resilient durable and elastic material that conforms to the human hand.

In one embodiment, a palm wrap device for providing support for the wrist of a user, comprises a top surface and a bottom surface, wherein the bottom surface comprises a palm pocket; one or more palm wedges inserted into said palm pocket; a first protruding tab and a second protruding tab; and a plurality of fasteners.

In another embodiment, the palm wrap device comprises elastic material.

In yet another embodiment, the palm pocket is attached to the bottom surface via a hook-and-loop tape, fabric glue, adhesive, lamination or by stitching sewn along outer edges of the palm pocket. In an embodiment, the bottom surface comprises a plurality of palm pockets positioned parallel to each other. And in another embodiment, the device further comprises a wrist support band that is partially attached along the bottom surface of the wrap device by sewing, or gluing.

In yet another embodiment, the plurality of palm pockets comprises parallel spacing about 1 cm to 3 cm apart.

In a further embodiment, the palm wrap device for providing support for the wrist of a user, comprises a top surface and a bottom surface, wherein the bottom surface comprises a palm pocket; one or more palm wedges inserted into the palm pocket; a first protruding tab and a second protruding tab; a plurality of first fastener affixed to the bottom surface of the protruding tab; a second, fastener affixed to the bottom surface of the second protruding tab; a third fastener affixed to the top surface of the second protruding tab; and a fourth fastener affixed to the top surface of the broad end.

In another embodiment, the palm pocket is attached to the bottom surface via a hook-and-loop tape, fabric glue, adhesive, lamination or by stitching sewn along outer edges of the palm pocket. The bottom surface comprises a plurality of palm pockets positioned parallel to each other. In another embodiment, the device further comprises a wrist support band that is partially attached along the bottom surface of the wrap device by sewing, or gluing. In yet another embodiment, the support band comprises elastic material configured to wrap securely around the user's wrist by hook-and-loop tape. In an embodiment, the plurality of palm pockets comprises parallel spacing about 1 cm to 3 cm apart. In an embodiment, the protruding tab is positioned and fastened over the top of the second protruding tab.

In yet another embodiment, the first and the second protruding tab are positioned around the hand of the user and are attached to the broad end.

In still another embodiment, the palm wrap device for providing support for the wrist of a user, comprises a top surface and a bottom surface, wherein the bottom surface comprises a palm pocket; one or more palm wedges inserted into the palm pocket; a first protruding tab and a second protruding tab, wherein the first protruding tab is positioned and fastened over the top of the second protruding tab; a plurality of first fastener affixed to the bottom surface of the protruding tab; a second, fastener affixed to the bottom surface of the second protruding tab; a third fastener affixed to the top surface of the second protruding tab; a fourth fastener affixed to the top surface of the broad end; and a wrist support band attached to the bottom surface of the wrap device; wherein the first and the second protruding tab are positioned around the hand of the user and are attached to the broad end. The palm wrap device according to claim 15, wherein the palm wrap device comprises elastic material.

In an embodiment, the palm pocket is attached to the bottom surface via a hook-and-loop tape, fabric glue, adhesive, lamination or by stitching sewn along outer edges of the palm pocket.

In another embodiment, the bottom surface comprises a plurality of palm pockets positioned parallel to each other.

In yet another embodiment, the plurality of the palm pockets comprises parallel spacing about 1 cm to 3 cm apart.

In one embodiment, the palm wrap device contains a palm pocket attached to the bottom surface of the device using fabric glue or other suitable adhesive. In another embodiment, the palm pocket is attached to the bottom surface using hook-and-loop tape. In another embodiment, the palm pocket is attached to the bottom surface using lamination. An exemplary embodiment of the device contains a rectangular shaped palm pocket attached to the bottom surface by sewing along the outer edges.

The palm wedge may vary in size and shape to further assist with the different techniques associated with a deep tissue massage. An embodiment of the device provides a palm wedge which is used in connection with the palm pocket. In another embodiment, one or more palm wedge(s) is placed in the opening of the palm pocket. In one embodiment, the palm wedge comprises non-slip tape overs. In yet another embodiment, the palm wedge comprises hard rubber-like material. In another embodiment, the palm wedge is shaped similar to a human thumb.

In one embodiment, the device comprises a first protruding tab, a second protruding tab and a broad end which fastens around the hand in a stationary position. The first fastener is attached to the bottom surface of the first protruding tab. The second fastener is attached to the bottom surface of the second protruding tab. The third fastener is attached to the top surface of the second protruding tab. The fourth fastener is attached to the top surface of the broad end. In another embodiment, the first protruding tab and the broad end extend in opposite directions and connect to the second protruding tab.

In an embodiment, the material of construction for the first, second, third, and fourth fasteners may consist of hook-and-loop tape. In another embodiment, the material of construction for the first, second, third, and fourth fastener is lamination. In yet another embodiment, the material of construction for the first, second, third, and fourth fastener is adhesive.

In another embodiment, the first protruding tab and second protruding tab are attached to the broad end by stitching sewn along the outer edges. In an exemplary embodiment, the first protruding tab stretches opposite from the broad end and is removably positioned above the thumb. In the same embodiment, the second protruding tab is removably positioned around the hand and below the thumb. In a further embodiment, the first and second protruding tabs provide support for the hand.

In another embodiment, the second protruding tab is positioned and fastened over the top of the first protruding tab. In the same embodiment, both the first protruding tab and the second protruding tab are positioned and fastened over the top of the broad end. The palm wrap device may be worn on either the left or right hand. In one embodiment, the device comprises an optional wrist support band comprising elastic type material. In an embodiment, the wrist support band provides added strength and support to the wrist.

In one embodiment, the wrist support band is partially attached along the bottom surface of the device by sewing. In another embodiment, the wrist support band is partially attached along the bottom surface of the device by glue. In yet another embodiment, the wrist support band is partially attached along the bottom surface of the device by hook-and-loop tape. In another embodiment, the wrist support band is attached by a durable strip of leather cord. The cord is attached to the top surface of the device and is about a length of 1 to about 2 inches to allow for comfortable wrist movement while massaging. In one embodiment, the wrist support band is about 1.5 to about 3.25 inches in width. In yet another embodiment, the wrist support band is about 2.25 inches in width of elastic material that wraps around the wrist in the form of a band and is secured to the wrist with hook-and-loop tape.

Various objects, features, aspects and advantages of the inventive subject matter will become more apparent from the following detailed description of exemplary embodiments, along with the accompanying figures in which like numerals represent like components.

### BRIEF DESCRIPTION OF THE DRAWINGS

FIG. 1 depicts the top surface of an exemplary configuration of a palm wrap device.

FIG. 2 depicts the bottom surface of an exemplary configuration of a palm wrap device.

FIG. 3 depicts an exemplary configuration of the posterior view of a palm wrap device wrapped to the user's hand.

FIG. 4 depicts an exemplary configuration of the anterior view of a palm wrap device wrapped and fastened to the user's hand.

FIG. 5 depicts an exemplary configuration of the anterior view of a palm device wrap, along with an optional wrist support band, wrapped and fastened to the user's hand.

FIG. 6 depicts an exemplary configuration of a posterior view of a palm device wrap, along with an optional wrist support band both wrapped and fastened to the user's hand.

FIG. 7 depicts an exemplary configuration of the top view of palm wedges.

FIG. 8 depicts an exemplary configuration of the bottom surface of a palm wrap device, along with a palm wedge embedded in the palm pocket.

### DETAILED DESCRIPTION

A palm wrap device is designed and manufactured such that it provides support for the wrist; protects against sprains of the wrist; and relieves tension during massages by serving as a deep tissue thumb massager. A palm wrap device is configured to keep the fingers and thumbs of a user exposed to assist the user with identifying tension knots and muscles using his or her fingertips.

Furthermore, as used herein, and unless the context dictates otherwise, the term “massagist” is intended to include both laypersons and a professional masseuse or masseur. Therefore, the terms “massagist”, “layperson”, “masseuse” and “masseur” are used synonymously.

As used in the description herein and throughout the claims that follow, the meaning of “a” “an”, “and”, and “the” includes plural reference unless the context clearly dictates otherwise. Also, as used in the description herein, the meaning of “in” includes “into” and “on” unless the context clearly dictates otherwise.

As used herein, the term “about” in conjunction with a numeral refers to a range of that numeral starting from 10% below the absolute of the numeral to 10% above the absolute of the numeral, inclusive.

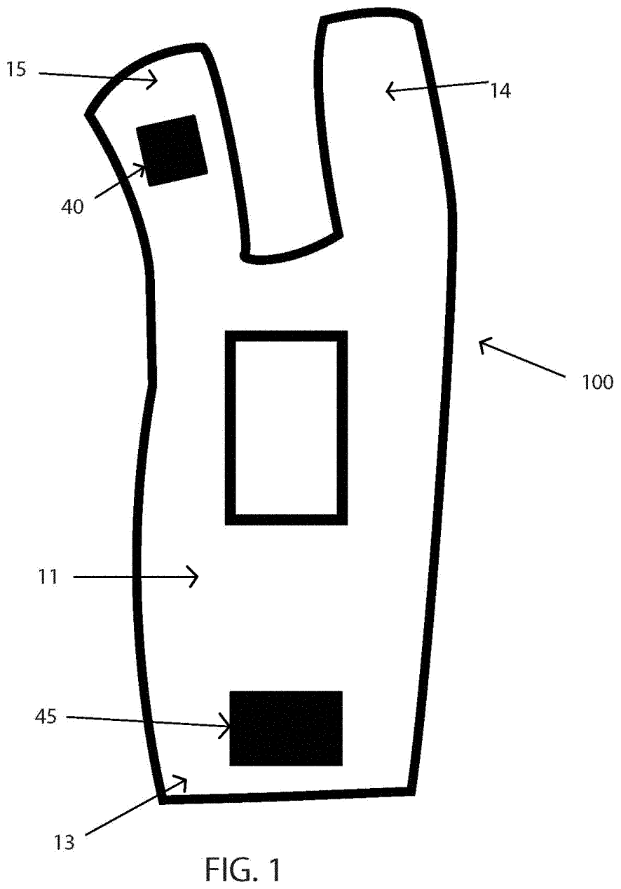

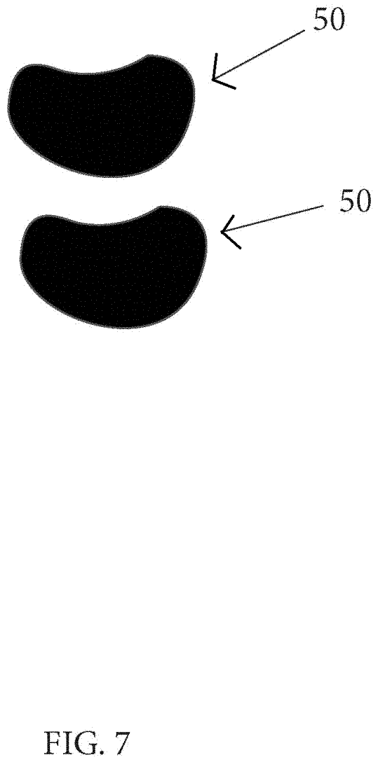

Exemplary configurations of the present invention are depicted in FIGS. 1-8, in which a palm wrap device 100 is configured to be worn on massaging hand 60 and wrist 70 of a user. In one embodiment, palm wrap device 100 comprises top surface 11, bottom surface 12, broad end 13, first protruding tab 14, and second protruding tab 15. In an embodiment, bottom surface 12 comprises palm pocket 10. In one embodiment, palm wrap device 100 comprises first fastener 20 affixed to bottom surface of first protruding tab 14. In an embodiment, palm wrap device comprises second fastener 30 affixed to bottom surface of second protruding tab 15 and third fastener 40 affixed to top surface of second protruding tab 15. In another embodiment, fourth fastener 45 is affixed to top surface of broad end 13.

In an embodiment, palm pocket 10 comprises one or more palm wedges 50. In an embodiment, device 100 comprises optional wrist support band 90 for additional support for wrist 70. In another embodiment, bottom surface 12 comprises one or more palm pockets 10 positioned parallel to each other (not shown) spacing about 1 cm to 3 cm apart. One of ordinary skill in the art can envision other arrangements for palm pocket 10 that are within the scope of the present invention.

In one embodiment, material of construction for device 100 comprises resilient durable material with the ability to conform to massaging hand 60 with the use of elastic material such as neoprene, polyester, polypropylene or wool, and any combination thereof (see FIG. 1). In yet another embodiment, the material of construction for device 100 comprises medium to thick leather. In another embodiment, thickness of material of construction for device 100 is about 3 mm to about 4 mm. A skilled person in the art will appreciate other types of material of construction for device 100.

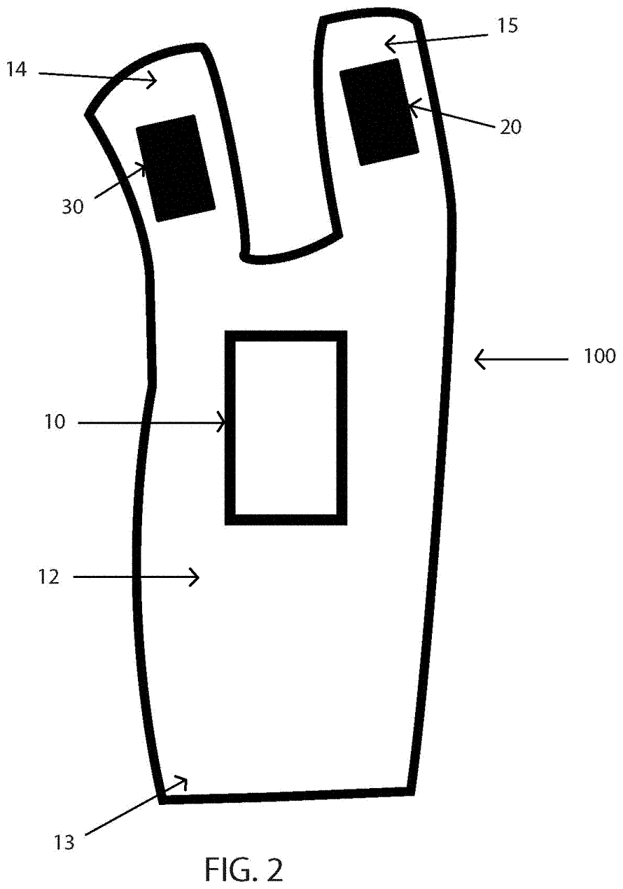

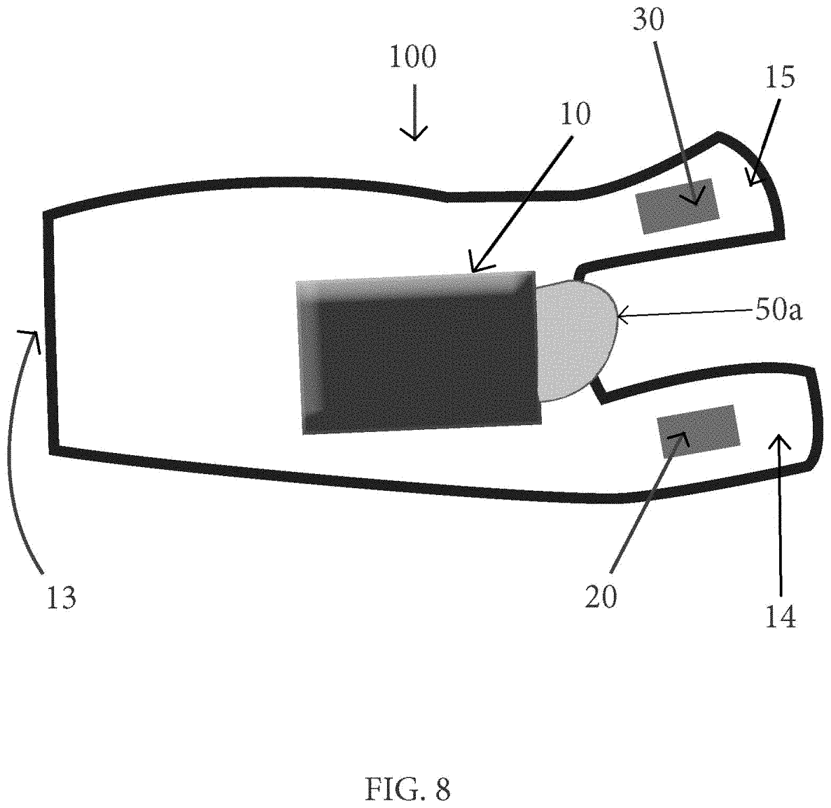

In an exemplary embodiment, palm pocket 10 comprises a rectangular, oval, or square shape, or any combination thereof. (see FIGS. 2 and 8). A skilled person in the art will be readily able to select an alternative suitable shape for palm pocket 10 based upon operating conditions and requirements in the configurations contemplated herein.

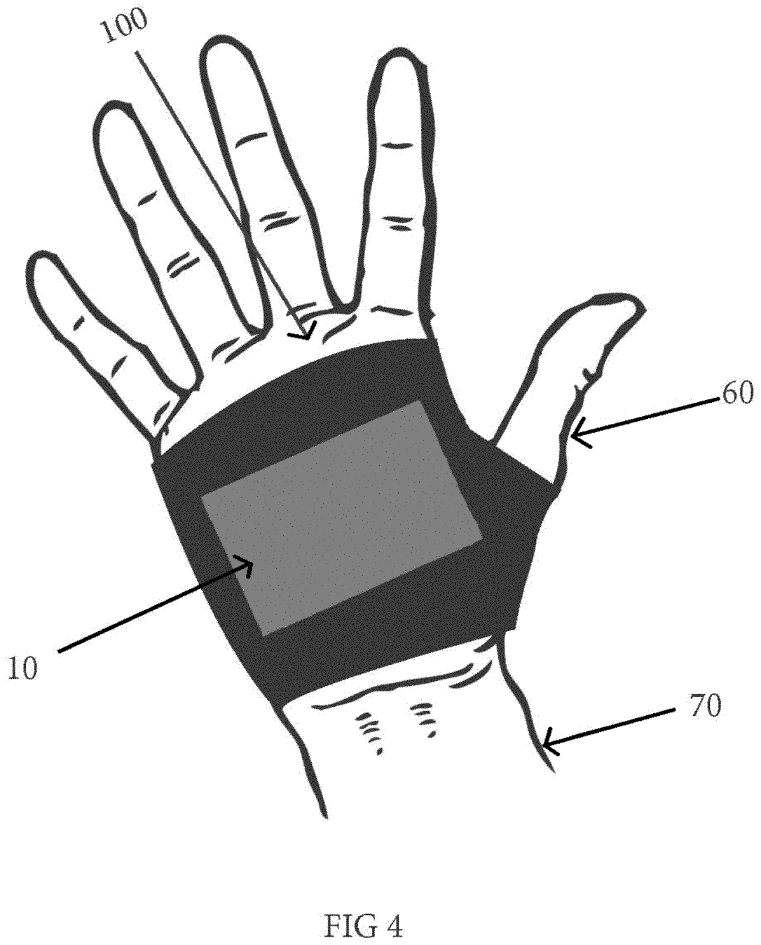

In an exemplary embodiment, material of construction for the palm pocket 10 comprises an elastic and nylon blend or any combination thereof. In another embodiment, palm pocket 10 material is durable in nature and strong (see FIG. 4). In an embodiment, palm pocket 10 comprises silk, linen or cotton knit fabric, or any combination thereof. In one embodiment, palm pocket 10 is attached to device 100 using fabric glue, hook-and-loop tape, adhesive, lamination, or by stitching sewn along the outer edges of palm pocket 10.

An exemplary configuration is depicted in FIG. 8, in which palm wedge 50 is used in connection with palm pocket 10. In an embodiment, palm wedge 50 comprises non-slip tape covers to secure one or more palm wedges 50 onto the palm pocket 10. In yet another embodiment, palm wedge 50 comprises hard rubberlike material that is not easily deformable by pressure applied in use.

In an embodiment, palm wedge 50 comprises a shape and size similar to a human thumb (see FIG. 8) and serves as a thumb block 50a. In one embodiment, palm wedge 50 is positioned within the opening of palm pocket 10 (as shown in FIG. 8) where it remains relatively stationary during the course of the deep tissue massage.

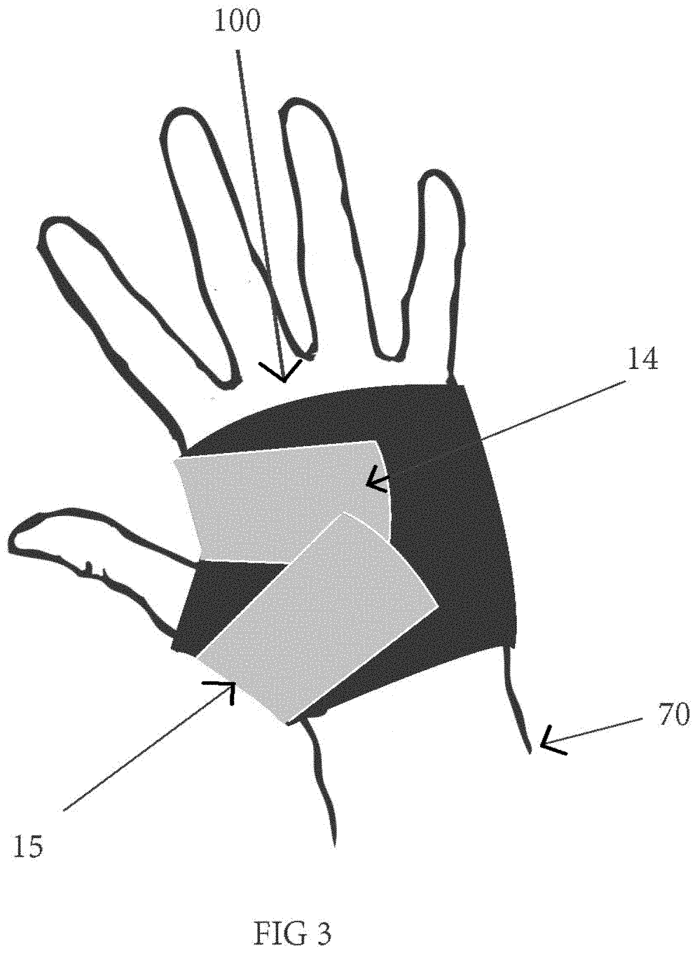

As shown in FIGS. 1, 2, and 8, first fastener 20, second fastener 30, third fastener 40, and fourth fastener 45 comprise hook-and-loop tape or any other suitable combination thereof for fasteners that are configured to hold wrap device 100 together. In an exemplary embodiment, broad end 13 is placed on the back of the hand and first protruding tab 14 and second protruding tab 15 extend around the palm of the hand and attach to third fastener 40, and fourth fastener 45 (as shown in FIG. 3). In one embodiment, first fastener 20, second fastener 30, third fastener 40, and fourth fastener 45 (see FIG. 1), are affixed to wrap device 100 by any adequate method including, but not limited to, sewing, lamination, or adhesive.

An embodiment in FIG. 3 also illustrates first protruding tab 14 and second protruding tab 15 connected by alternative and suitable means across the back of hand 60 to affix to broad end 13. In an embodiment, first protruding tab 14 is removably positioned above the thumb (as shown in FIGS. 3-6) and second protruding tab 15 is removably positioned below the thumb to secure device 100 around hand 60.

In an exemplary embodiment, first protruding tab 14 and second protruding tab 15 provide support for hand 60 from different directions. In another embodiment, first protruding tab 14 is positioned and fastened over top of second protruding tab 15. In yet another embodiment, both first protruding tab 14 and second protruding tab 15 are positioned and wrapped around hand 60 and attach to broad end 13.

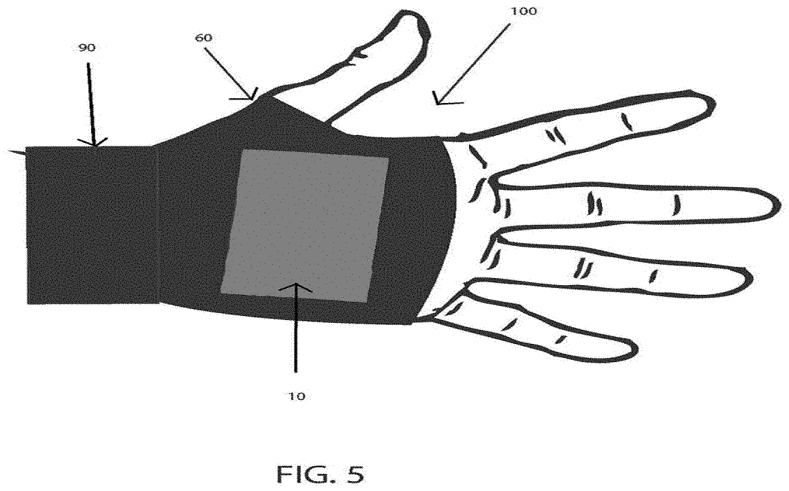

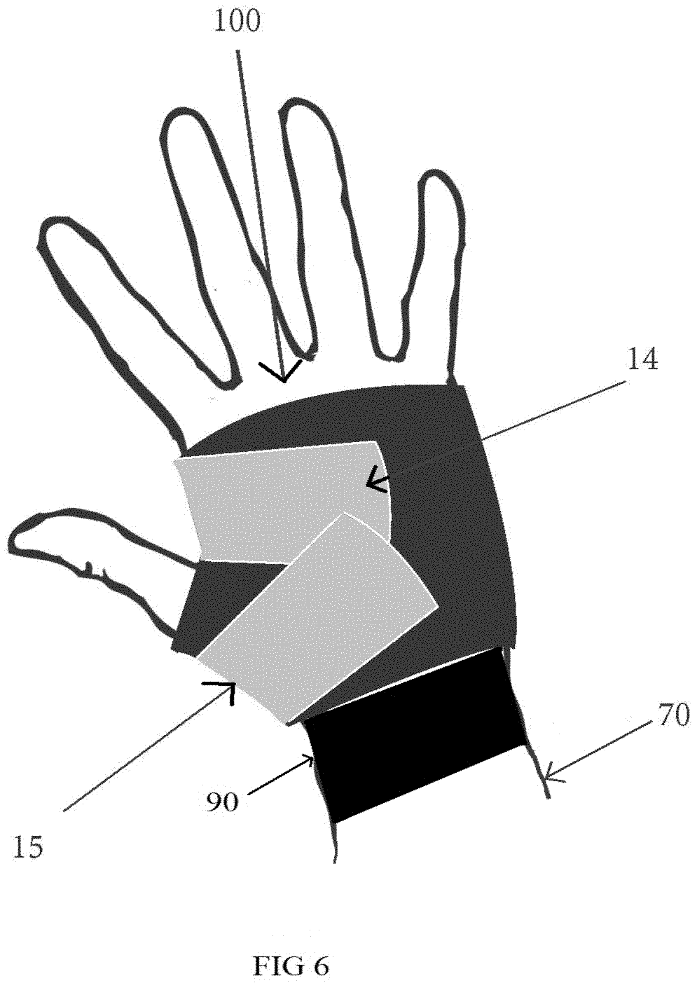

As illustrated in FIGS. 5 and 6, palm wrap device 100 comprises an optional wrist support band 90 that comprises elastic material for protection against wrist sprains, providing additional strength and support to a user's wrist 70. In an exemplary embodiment, wrist support band 90 is partially attached along bottom surface 12 of wrap device 100 by method of sewing, gluing, or other suitable means of attachment. In an embodiment, wrist support band 90 is about 1.5 inches to about 3.25 inches wide. In another embodiment, wrist support band 90 is about 1.25 inches to about 2.25 inches wide. In another, wrist support band 90 is about 1 inch to about 3 inches wide. One of ordinary skill in the art will appreciate other dimensions for wrist support band 90 that is in accordance with the present invention and accommodates the user's needs. In an embodiment, wrist support band 90 comprises elastic material configured to wrap firmly around wrist 70 and is secured with hook-and-loop tape or other means of suitable attachment by the massagist.

Thus, specific embodiments of a palm wrap device to assist with deep tissue massaging and methods to employ such device have been disclosed. It should be apparent, however, to those skilled in the art that additional modifications besides those already described are possible without departing from the inventive concepts herein. Moreover, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. A palm wrap device for providing support for the wrist of a user, comprising:
(a) a top surface and a bottom surface, wherein said bottom surface comprises a palm pocket;
(b) one or more palm wedges inserted into said palm pocket;
(c) a first protruding tab and a second protruding tab; and
(d) a plurality of fasteners.

2. The palm wrap device according to claim 1, wherein said palm wrap device comprises elastic material.

3. The palm wrap device according to claim 1, wherein said palm pocket is attached to said bottom surface via a hook-and-loop tape, fabric glue, adhesive, lamination or by stitching sewn along outer edges of said palm pocket.

4. The palm wrap device according to claim 1, wherein said bottom surface comprises a plurality of palm pockets positioned parallel to each other.

5. The palm wrap device according to claim 1, wherein said device further comprises a wrist support band that is partially attached along said bottom surface of said wrap device by sewing, or gluing.

6. The palm wrap device according to claim 1, wherein said plurality of palm pockets comprise parallel spacing about 1 cm to 3 cm apart.

7. A palm wrap device for providing support for the wrist of a user, comprising:
(a) a top surface and a bottom surface, wherein said bottom surface comprises a palm pocket;
(b) one or more palm wedges inserted into said palm pocket;
(c) a first protruding tab and a second protruding tab;
(d) a plurality of first fastener affixed to the bottom surface of said protruding tab;
(e) a second, fastener affixed to the bottom surface of said second protruding tab;
(f) a third fastener affixed to the top surface of said second protruding tab; and
(g) a fourth fastener affixed to the top surface of said broad end.

8. The palm wrap device according to claim 7, wherein said palm wrap device comprises elastic material.

9. The palm wrap device according to claim 7, wherein said palm pocket is attached to said bottom surface via a hook-and-loop tape, fabric glue, adhesive, lamination or by stitching sewn along outer edges of said palm pocket.

10. The palm wrap device according to claim 7, wherein said bottom surface comprises a plurality of palm pockets positioned parallel to each other.

11. The palm wrap device according to claim 7, wherein said device further comprises a wrist support band that is partially attached along said bottom surface of said wrap device by sewing, or gluing.

12. The palm device according to claim 11, wherein said wrist support band comprises elastic material configured to wrap securely around said user's wrist by hook-and-loop tape.

12. The palm wrap device according to claim 7, wherein said plurality of palm pockets comprise parallel spacing about 1 cm to 3 cm apart.

13. The palm device according to claim 7, wherein said first protruding tab is positioned and fastened over the top of said second protruding tab.

14. The palm device of claim 7, wherein said first and said second protruding tab are positioned around the hand of said user and are attached to said broad end.

15. A palm wrap device for providing support for the wrist of a user, comprising:
(a) a top surface and a bottom surface, wherein said bottom surface comprises a palm pocket;
(b) one or more palm wedges inserted into said palm pocket;
(c) a first protruding tab and a second protruding tab, wherein said first protruding tab is positioned and fastened over the top of said second protruding tab;
(d) a plurality of first fastener affixed to the bottom surface of said protruding tab;
(e) a second, fastener affixed to the bottom surface of said second protruding tab;
(f) a third fastener affixed to the top surface of said second protruding tab;
(g) a fourth fastener affixed to the top surface of said broad end; and
(h) a wrist support band attached to said bottom surface of said wrap device;
wherein said first and said second protruding tab are positioned around the hand of said user and are attached to said broad end.

16. The palm wrap device according to claim 15, wherein said palm wrap device comprises elastic material.

17. The palm wrap device according to claim 15, wherein said palm pocket is attached to said bottom surface via a hook-and-loop tape, fabric glue, adhesive, lamination or by stitching sewn along outer edges of said palm pocket.

18. The palm wrap device according to claim 15, wherein said bottom surface comprises a plurality of palm pockets positioned parallel to each other.

19. The palm wrap device according to claim 15, wherein said plurality of palm pockets comprise parallel spacing about 1 cm to 3 cm apart.
